(19) United States
(12) Patent Application Publication
(10) Pub. No.: US 2025/0162985 A1
(43) Pub. Date: May 22, 2025

(54) COMPOSITION AND PROCESS OF ELABORATION OF NON-HYDROLYZABLE SURFACTANTS FROM VEGETABLE OILS

(71) Applicant: CORROSION Y PROTECCION S.A. DE C.V., Mexico (MX)

(72) Inventors: Jorge Joaquin Canto Ibanez, Mexico (MX); Lorenzo Martinez Martinez De La Escalera, Mexico (MX); Adrian Cristobal Neri, Mexico (MX); Abraham Matla Baez, Mexico (MX); Guillermo Barrera Sanchez, Mexico (MX); Jose Luis Montelongo Villar, mexico (MX)

(73) Assignee: CORROSION Y PROTECCION S.A. DE C.V., Mexico (MX)

(21) Appl. No.: 18/516,183

(22) Filed: Nov. 21, 2023

(51) Int. Cl. C07C 303/32; C07C 29/147; C07C 41/03; C07C 51/41; C07C 209/50; C07C 213/00; C07C 231/02; C07D 301/24; C09K 8/035; C11C 3/00

(52) U.S. Cl. CPC C07C 303/32; C07C 29/147; C07C 41/03; C07C 51/412; C07C 209/50; C07C 213/00; C07C 231/02; C07D 301/24; C09K 8/035; C11C 3/003

(57) ABSTRACT

Chemical additives for the stimulation of oil wells and their formulations. The invention discloses a micelle-forming surface tension modifying agent, as well as synthesis processes, to be used to modify rock permeability, which supports pressure and temperature under well conditions. The formulation has as an additional advantage, that it is made up, in most of its components, with ingredients derived from natural origin and that it does not present hydrolysis phenomenon at the well working temperature, it is highly compatible with high pressure and it can be recover for a reinjection. The invention can be used in different proportions of formulations for stimulating oil wells.

### TECHNICAL FIELD

The present invention belongs to the technical field of chemistry. Particularly to the technical field of emulsifying, wetting, dispersing or foaming agents and more particularly to the technical field of surfactants or surfactants.

### BACKGROUND OF THE INVENTION

Surfactants, also known as surfactants, are chemical compounds that are used in industrial products, such as soaps, detergents, cosmetics, emulsifiers, drugs, among others, and in some processes in the petrochemical and food industries. These compounds have the ability to affect the surface and interface properties of liquids, which has earned them the name “surface active agents.” Surfactants or surfactants are chemical substances of an amphiphilic nature that have the ability to alter the properties at the interface of a fluid. The interface is defined as the boundary between two liquid phases. When dealing with a solid-liquid pair, the surfactant modifies the wettability of the liquid on the solid; if the contact angle θ is less than 90°, the wettability is high.

These chemical compounds that are used in a wide range of industrial products, as well as in petrochemical and food industry processes and have the property of altering the surface and interface properties of liquids, and are therefore called “agents” surface assets. Surfactants are classified according to the electrical charge of their head group in aqueous solution. In the state of the art, some authors classify them into four groups (anionic, cationic, non-ionic, amphoteric) and other authors classify them into five groups. anionic, cationic, non-ionic, amphoteric and catanionic (formed by the union of a cationic surfactant and an anionic one that acts as a counterion).

Sulfate polar segment surfactants are thermolabile and easily hydrolyze with increasing temperature and acidic media. The function of the surfactant is to decrease the contact angle between the two immiscible liquids, in order to form a micelle and thus achieve an emulsification.

The use of surfactants has been extensive in the field of hydrocarbon extraction, making it possible to push or drag oil impregnated in the rocks; and take it to the producing well. The conditions inside a well are characterized as extreme; internal temperature can reach 100° C., pressure ranges from 2,000 to 5,000 pounds per square inch, pH is generally alkaline due to numerous carbonate deposits, however this value can lean towards acidic due to additives chemicals used as hydrochloric, hydrofluoric, acetic, formic, fluoroboric acid, among others.

Various proposals for obtaining surfactants with uses in the extraction of hydrocarbons are known in the state of the art, such as U.S. Pat. No. 5,042,580 A, which provides a process for recovering oil from fractured formations that involves altering the wettability of the formation, particularly at the interface of the fracture and the rock matrix. The process of the present invention improves the ability of injected fluids flowing in the fracture to enter the rock matrix and displace oil. When used in conjunction with a water-wet fractured petroleum formation, the process involves the steps of: injecting a wettability agent capable of transforming the water-wet fractured formation from water-wet to oil-wet; contacting the oil-wet fractured formation with the wettability agent so injected at a fracture/matrix interface; injecting an oil-miscible solvent; and oil recovery. A process for use with an oil-wet fractured petroleum formation is also provided.

US application US20190382644 A1, which discloses an enhanced oil recovery method in which: (a) a flooding composition is delivered in an underground reservoir; (b) the flooding composition includes a sulfur surfactant; and (c) the fluid produced from the underground reservoir can also be analyzed to determine if surfactant is present in the fluid. The surfactant preferably includes a sulfonate or other sulfur-containing moiety.

In the application US2009023618 A1, an aqueous fluid useful for the recovery of crude oil from an underground formation is mentioned, which includes water and one or more organophosphorus materials and methods for using them.

Another case is that described in the European application EP 2644685 A1, which proposes the application of geminal zwitterionic liquids based on bis-N-alkyl or bis-N-alkenyl or N-cycloalkyl or N-aryl bis-beta amino acids or their salts. as rock wettability modifiers in the presence of brines with a high content of divalent ions, high temperature and pressure in enhanced oil recovery processes. Geminal zwitterionic liquid based on bis-N-alkyl or bis-N-alkenyl or N-cycloalkyl or N-aryl bis-beta amino acids or their salts are obtained by reacting an alkyl or alkenyl amine and a polyether oxide reagent derivative ethylene or propylene. or copolymer of these, which may contain mesyl or tosyl groups in their structure,

### OBJECT OF THE INVENTION

The present invention refers to a chemical process for the preparation of non-hydrolyzable surfactants from vegetable oils; In a particular modality, the surfactants are obtained starting from a fatty alcohol, for example, dodecanol. Surprisingly, in the present invention, the synthesis route for non-hydrolyzable surfactants presents 2 alternative routes to generate the fatty alcohol from a vegetable oil.

Therefore, the main object of the present invention is a chemical synthesis process for obtaining non-hydrolyzable surfactants characterized in that it has the following stages:
vegetable oils are treated with potassium hydroxide and methanol, for the formation of the mixtures of methyl esters of the corresponding fatty acids;
the methyl esters, from the previous stage, are treated with sodium borohydride (NaBH4), tetrahydrofuran (TFH) in methanol to generate the corresponding fatty alcohol mixture;
the fatty alcohols, from the previous stage, are treated with zinc chloride and epichlorohydrin to generate the corresponding fatty acid chlorohydrin mixture; at the same time
the fatty acid chlorohydrin mixture is treated with potassium hydroxide in methanol to generate the fatty acid epoxide mixture, and

Chemical synthesis is directed from the epoxide to obtain anionic, cationic or non-ionic non-hydrolyzable surfactants.

Still a second object of protection refers to a chemical synthesis process to obtain non-hydrolyzable surfactants characterized in that it presents the following stages:
vegetable oils are treated with dry ammonia dissolved in methanol and NaCN as a catalyst for the formation of the amide mixtures of the corresponding fatty acids;
the amides, from the previous stage, are treated with anhydrous tetrahydrofuran (TFH), sodium borohydride (NaBH4) and acetic acid, kept stirring for 20 min at room temperature; a THF/AcOH (4:1) mixture is added. The mixture is brought to reflux, the pH is adjusted between 1 and 2 and K2CO3 is added to obtain the alkylamine mixture;
the alkylamines, from the previous stage, are treated with formic acid to generate ammonium formates, formaldehyde is added, it is allowed to react at 70° C. until the reaction is complete, a saturated NaHCO3 solution is added until a pH of 8 is obtained, and

The product from the previous stage is treated with sodium chloroacetate and methanol at a pressure of 20 psi for 30 min, the pressure is released and it is heated at 110° C. for 6 hrs. to generate amphoteric non-hydrolyzable surfactants

An additional object of protection refers to the non-hydrolyzable surfactant characterized in that it is obtained in accordance with any of the synthesis processes described above, which is selected from the following group: 2,2′-((3-dodecyloxy)-2-hydroxypropyl) azadienyl)bis(ethan-1-ol); N,N-bis(2-aminoethyl)-3-(dodecyloxy)-2-hydroxypropan-1-ammonium; Sodium 3-dodecyloxy-2-hydroxypropan-1-sulfonate, 2-dodecyldimethylammonium acetate, and any combination of these.

Still another object of protection refers to a drilling fluid composition characterized in that it comprises one or more of the surfactants of the previous claim, one or more of a solvent and one or more of an additive.

The aforementioned objectives of the present invention and even others not mentioned will be evident from the description of the invention and the accompanying illustrative and non-limiting figures, which are presented below.

### BRIEF DESCRIPTION OF THE FIGURES

FIG. 1. General scheme of chemical synthesis for non-hydrolyzable surfactants (I, II, III and IV) from vegetable oils

FIG. 2. General scheme of chemical synthesis for non-hydrolyzable surfactants (6) from fatty alcohols.

FIG. 3. General chemical synthesis scheme for amphoteric (IV) NO hydrolyzable surfactants from vegetable oils.

FIG. 4. Structures of sodium borohydride (A), sodium acetoborohydride (B), sodium triacetoborohydride (C), the arrows indicate the direction of electron density displacement, the end of the arrow that has a perpendicular cross line indicates the positive or unprotected region of the structure.

FIG. 5. Analysis of the general mixture of alkylamines by gas chromatography. The peak with a retention time of 13.22 minutes (a) corresponds to 12-carbon amines, the peak that appears at 15.70 minutes (b) corresponds to 14-carbon amines, while the 17.97 (c) peak corresponds to amines of 18 carbons.

FIG. 6. Gas-mass chromatography for product 3, mixture of fatty alcohols.

FIG. 7A. Mass spectrum of the dodecanol compound theoretical.

FIG. 7B Mass spectrum of the dodecanol compound experimental.

FIG. 8. 1H NMR spectrum, low resolution, for product 4, mixture of fatty acid chlorohydrins.

FIG. 9. 1H NMR spectrum, low resolution, for product 5, mixture of fatty acid epoxides.

FIG. 10. Low resolution 1H NMR spectrum for product 6, anionic surfactant.

FIG. 11. 1H NMR spectrum, low resolution, magnification for product 6, anionic surfactant.

FIG. 12. Electrospray ionization spectrum (ESI-MS) for product I, Calculated molecular weight 323.47 g/mol, obtained molecular weight 323.0 g/mol.

FIG. 13. Critical micellar concentration.

FIG. 14. Reversed phase chromatographic analysis for the anionic surfactant obtained. Chromatogram 1 shows the reference test, chromatogram 2 shows the experimental test, after subjecting the anionic surfactant to critical working conditions.

FIG. 15. Reversed phase chromatographic analysis for the cationic surfactant, obtained. In chromatogram 1 the reference test is shown, in chromatogram 2 the experimental test is shown, after subjecting the cationic surfactant to critical working conditions.

FIG. 16. Reversed phase chromatographic analysis for nonionic surfactant obtained. In chromatogram 1 the reference test is shown, in chromatogram 2 the experimental test is shown, after subjecting the non-ionic surfactant to critical working conditions.

FIG. 17. Reversed phase chromatographic analysis for obtained zwitterionic surfactant. In the upper chromatogram the reference test is shown, in the lower chromatogram the experimental test is shown, after subjecting the zwitterionic surfactant to critical working conditions.

### DESCRIPTION OF THE INVENTION

Below, various synthesis routes for non-hydrolyzable surfactants from vegetable oils and compositions comprising said surfactants are presented.

In one modality, the chemical synthesis is designed to use vegetable oils as raw material (1), the ester reduction process is direct from the triglyceride to the alcohol mixture (3) by using a mild reducing agent in polar solvents. without the use of strong reducing agents such as lithium aluminum hydride or high-pressure catalytic hydrogenation.

When the epoxyalkyl (5) is obtained, the chemical synthesis can be directed to obtain anionic (I), cationic (II), and nonionic (III) surfactants; if switerionic (IV) surfactants are required, route B must be followed. This process generates non-hydrolyzable surfactants, chemical materials that are stable under working conditions in an oil well and useful for the well stimulation process, since they withstand temperatures up to 180° C. and pressure up to 4000 psi.

Below, various definitions of terms used in the present invention are provided, in the event that a term is not defined in this document then it must be given the conventional technical meaning in the technical area.

The term “vegetable oil” as used in the present invention refers to any vegetable oil that has a high percentage of triglycerides, including coconut, palm, palm kernel, jatropha curcas, castor, corn, canola, sunflower, soybean, birdseed, safflower, Karite, peanut, sesame, sunflower, cotton, etc.

The term “additive” as used in the present invention refers to any agent that is added to a surfactant composition for the purpose of improving oil recovery. Thus, an additive is selected from a group comprising a solvent, a cosolvent, a paraffin and asphaltene inhibitor, an iron sequestering agent, a dispersant, a corrosion inhibitor, an acid, a salt, an anti-sludge, a permeability improver, among others.

### General Synthesis Scheme a, Pure Surfactant

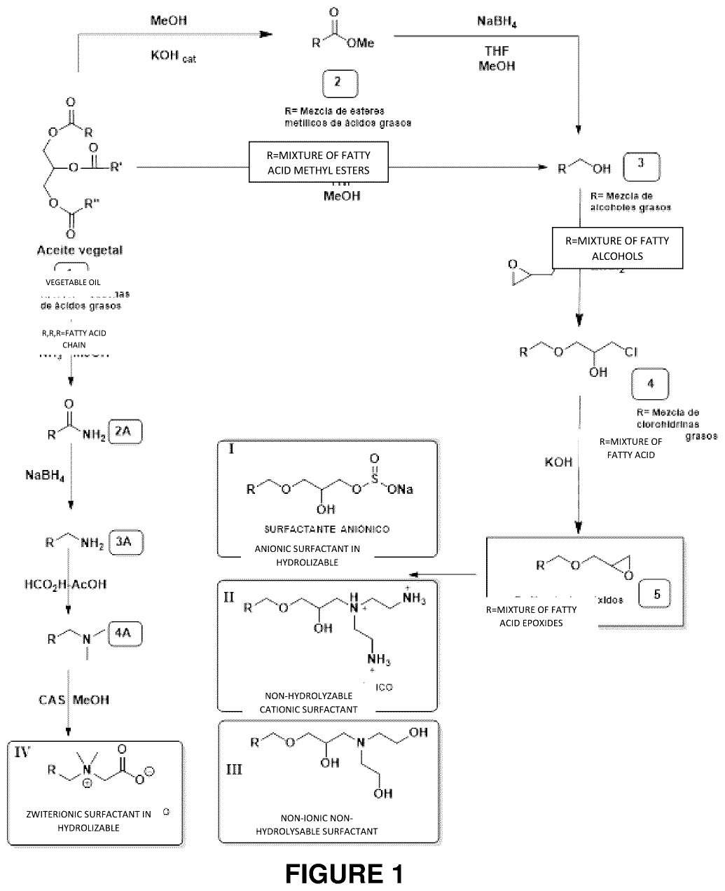

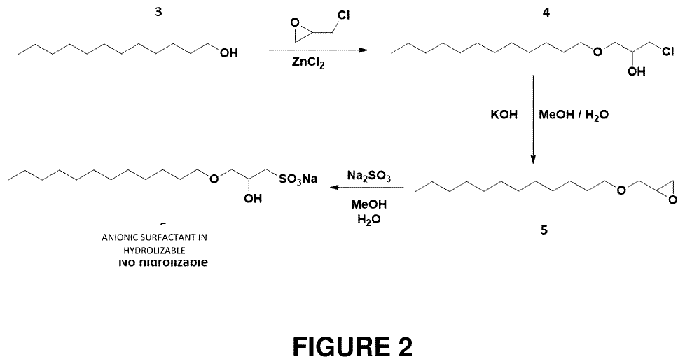

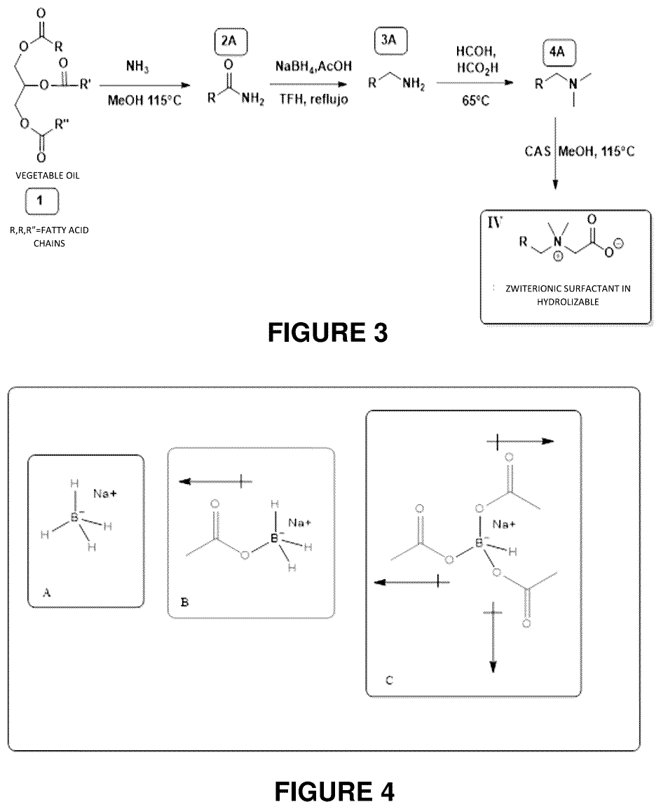

In general, the development of the synthesis of the compound 3-dodecyloxy-2-hydroxypropane-1-sulfonate from dodecanol (fatty alcohol) is described, but as shown in FIG. 1, this methodology was adapted for the synthesis of the surfactant mixture from a vegetable oil source, eg coconut oil or some other triglyceride (Synthesis Route B). FIG. 2 shows the route used for the pure surfactant and this method is used to obtain surfactants from vegetable oils. Compound (3) is a fatty alcohol that is converted to a chlorohydrin (4), followed by treatment with potassium hydroxide in methanol to obtain an epoxide (5) and finally ring opening with sodium sulfite in a mixture methanol-water, the product (6) is the NON-hydrolyzable surfactant,

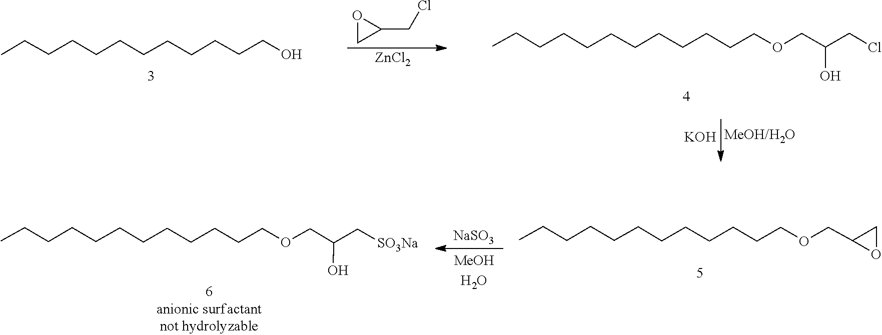

Next, the synthesis route of the pure NO hydrolyzable surfactant is described in detail: 3-dodecyloxy-2-hydroxypropane-1-sulfonate.

Obtaining the compound: 1-chloro-3-dodecyloxypropan-2-ol.

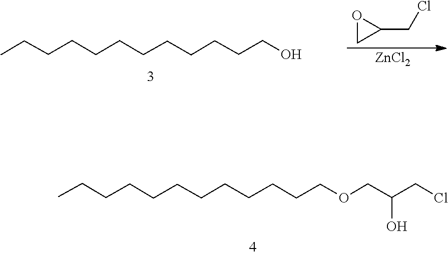

From a pure fatty alcohol (3), in a 250 mL three-necked balloon flask equipped with magnetic stirring, thermometer, addition funnel, 10 g (53.668 mmol) of dodecanol (3) with 160.93 mg (1.18 mmol) of ZnCl2 and heated at 60° C. for 10 minutes. 4.96 g (53.664 mmol) of epichlorohydrin were added dropwise over 1.5 hours, taking care that the temperature did not exceed 70° C. Once the addition was complete, it was kept at 65° C. for four hours, the progress of the reaction was verified by CCD (Heptane-AcOEt 80:20; run twice and developed with phosphomolybdic acid). 14 g of crude reaction mixture (4) were obtained and used without prior purification for the next reaction step. The performance is reported in table 1.

### Compound: 2-dodecyloxymethyloxirane

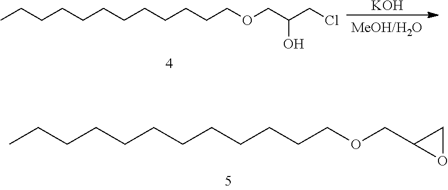

The previous reaction flask was placed in an ice bath and 18 mL of a 1.36 M KOH/MeOH solution were slowly added, shaken for 10 minutes, then removed from the bath to reach room temperature and left stirring for 6 hours. TLC (heptane-AcOEt (80:20)) was run twice and developed with sulfuric acid in water-methanol, adjusted to neutral pH with 450 μL of glacial AcOH. The reaction mixture was transferred to a separatory funnel and 45 ml of water were added and extracted with heptane (3×15 mL), the aqueous phase was discarded and the organic phase was dried over Na2SO4, concentrated in a rotary evaporator, obtaining 8.2 g of crude product (5). The performance is reported in table 1

Anionic Surfactant: Sodium 3-dodecyloxy-2-hydroxypropane-1-sulfonate.

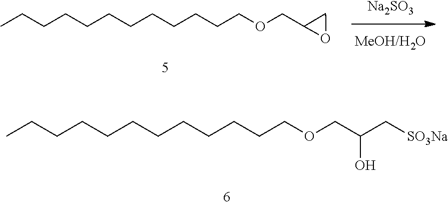

In a 250 mL Erlenmeyer flask, 2.6 g (20.63 mmol) of Na2SO3 were placed and dissolved with 120 mL of water, 180 mL of MeOH were added, obtaining a cloudy solution. This solution was placed in a high pressure reactor with vigorous stirring, and 5 g (20.63 mmol) of the epoxide (5) were added, the reactor was closed and heated at 95° C. for four hours, cooled to room temperature and dried. CCD was carried out (Heptane-AcOEt (80:20) twice) and revealed with phosphomolybdic acid, then the reactor was placed on an ice bath for two hours and vacuum filtered, the solid obtained was dried in a vacuum oven at 45° C. for 4 hours and 3.54 g of the sulfonate (6) were obtained. The performance is reported in table 1

### nonionic surfactantor: 2,2′-((3-dodecyloxy)-2-hydroxypropyl)azadienyl)bis(ethan-1-ol)

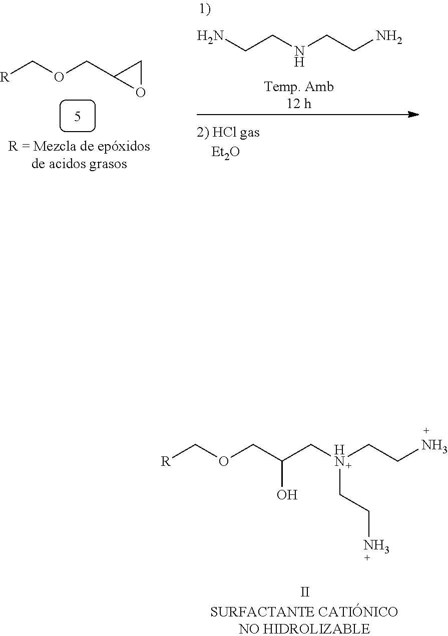

In a 250 mL balloon flask, 50 g of epoxide (5) were placed, kept stirring for 10 minutes, a septum cap was placed, purged with nitrogen and 27.3 g of diethylenetriamine were added slowly, left stirring for 1 hour, it was removed from the agitation, a representative sample was obtained to make a CCF plate in the DCM-MeOH—NH4OH phase (85:14:1), total consumption of raw material was observed. In a second step and in the same reaction flask, 20 mL of anhydrous ethyl ether were added via cannula, HCl gas was bubbled until white precipitates were obtained, at the end, the bubble was removed, filtered through paper for solids and under vacuum, obtained 42 g of cationic surfactant.

### cationic surfactant: N,N-bis(2-aminoethyl)-3-(dodecyloxy)-2-hydroxypropan-1-ammonium

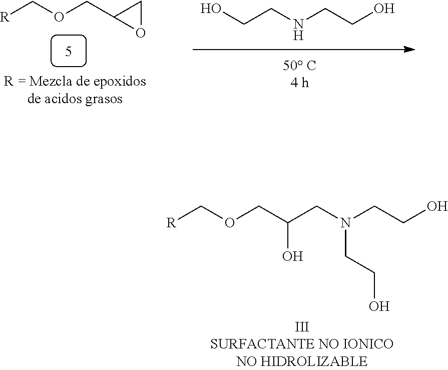

In a 250 mL balloon flask, 50 g of the previously synthesized epoxide (5) were placed, kept stirring for 10 minutes, and 22.4 g of diethanolamine were added slowly, a condenser was added and heated to 50° C. for 4 hours, a representative sample was obtained to make a CCF plate in the DCM-MeOH—NH4OH (85:14:1) phase, total consumption of raw material was observed. It was purified by a percolated column with 20 parts of silica gel in 100% dichloromethane phase, concentrated, and 38 g of liquid cationic surfactant were obtained.

### Synthesis Route B: Mixture of Surfactants

Next, route B for the synthesis of the NO-hydrolyzable surfactant from a vegetable oil is described in detail (1), see FIG. 1. This synthesis route shows the obtaining of a mixture of methyl esters (2) of fatty acids prior to obtaining the mixture of fatty alcohols (3).

### Mixture of Coconut Oil Methyl Esters (2)

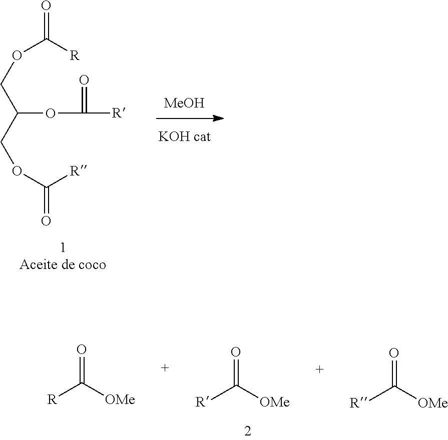

Firstly, the vegetable oils (1) are transesterified to generate the corresponding mixture of methyl esters (2) which, later, will give rise to the mixture of fatty alcohols (3) see FIG. 1.

50 g (77.12 mmol) of coconut oil (1) were placed in a 250 mL three-necked balloon flask equipped with magnetic stirring, water coolant, and heated at 80° C. for 15 minutes; subsequently, 20 mL of a 673 millimolar methanolic potassium hydroxide solution were added slowly (40 minutes). The mixture was heated at reflux for 1.5 hours and the end of the reaction was verified by TLC, Heptane-AcOEt (90:10). The reaction mixture was transferred to a separatory funnel and the glycerol was decanted, washed with 10% w/v NH4Cl solution (3×20 mL), added 50 ml of heptane and washed with water (2×30 mL). The aqueous phase was discarded and the organic phase was dried with anhydrous Na2SO4 and concentrated under reduced pressure, obtaining 43.5 g of crude product (2).

### Mixture of Coconut Spirits (3)

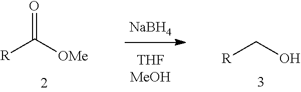

40 g of the methyl ester mixture (2) and 5.7 g (150.67 mmol) of NaBH4 were placed in a 500 mL three-necked balloon flask equipped with magnetic stirring, a thermometer, a water condenser, and an addition funnel. 200 mL THF, the mixture was heated at 50° C. for 10 minutes and 54 mL MeOH was added dropwise over two hours. At the end of the addition, it was left to heat at 50° C. for 1.5 hours and CCD (Heptane-AcOEt (90:10)) revealed with iodine was carried out to verify the consumption of methyl esters, later another CCD plate was carried out in phase Heptane-AcOEt (80:20) run twice and developed with phosphomolybdic acid to verify the formation of alcohol mixture. 250 ml of 10% NH4Cl solution were added and stirred for 30 minutes.

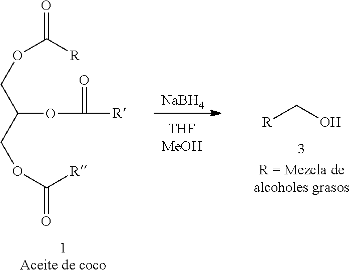

### Synthesis Route B′: Mixture of Fatty Alcohols

Next, a route B′ for the synthesis of the NO-hydrolyzable surfactant from a vegetable oil is described in detail (1), see FIG. 1. This alternative synthesis route shows the obtaining of the mixture of fatty alcohols (3) directly from vegetable oil.

Unlike the classical route of transesterification of vegetable oils (1) to generate methyl esters (2) and, subsequently, fatty alcohols (3), in the present synthesis route the synthesis of fatty alcohols is obtained directly. In a 1 L three-necked balloon flask equipped with magnetic stirring, thermometer, water condenser and addition funnel, 50 g of coconut oil expander (1) plus 300 mL of fresh THF were added and 15.7 g (415.01 mmol) of NaBH4, was heated at 50° C. for 10 minutes, then 130 ml of methanol were added dropwise over two hours and once the addition was complete, it was refluxed for two hours. CCD was carried out in the heptane-ethyl acetate (90:10) phase revealed with iodine, verifying the total consumption of coconut oil. Besides, Another plate was made in the heptane-AcOEt (80:20) phase, run twice and developed with phosphomolybdic acid, to verify the reduction towards alcohols. It was concentrated under reduced pressure and 100 mL of 0.5 N HCl solution were added, transferred to a separatory funnel and extracted with heptane (3×30 mL), the aqueous phase was discarded and the organic phase was dried over anhydrous Na2SO4, was concentrated obtaining 46.5 g of crude product 3.

### 1-alkoxy-3-chloropropan-2-ol (4)

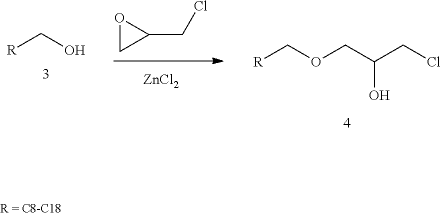

30 g (approx. 195 mmol) of the alcohol mixture (3) with 482.8 mg (3.5 mmol) of ZnCl2 were placed in a 250 mL three-necked balloon flask equipped with magnetic stirring, thermometer, and addition funnel and heated at 60° C., for 10 minutes. 17.88 g (193.2 mmol) of epichlorohydrin were added over 1.5 hours, taking care that the temperature did not exceed 70° C. Once the addition was complete, it was left at 65° C. for four hours, verifying the progress of the reaction by CCD (Heptane-AcOEt (80:20)) run twice and revealed with phosphomolybdic acid. 46 g of crude reaction mixture 4 were obtained

### 2-alkoxymethyloxirane (5)

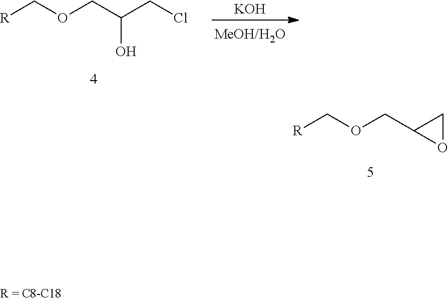

The above reaction flask was placed in an ice bath and 50 ml of a 1.36 M KOH/MeOH solution was slowly added, shaken for 10 min, removed from the bath to reach room temperature and allowed to stir for 6 hours. A CCD plate was made (heptane-AcOEt 80:20), adjusted to neutral pH with 1.8 mL of glacial AcOH, verified by potentiometer. The reaction mixture was transferred to a separatory funnel, 180 mL of water were added and it was extracted with heptane (3×60 mL), the organic phase was dried over Na2SO4 and concentrated under reduced pressure, obtaining 37.9 g of crude mixture. reaction (5) that was used without prior purification for the reaction to obtain sulfonates.

### 3-alkoxy-2-hydroxypropane-1-sulfonate (I)

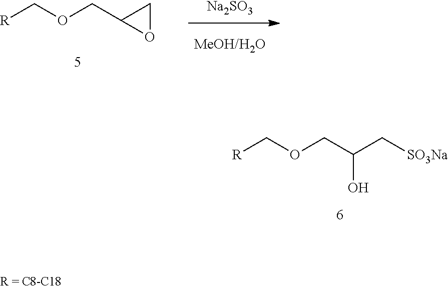

5.19 g (41.17 mmol) of Na2SO3 were placed in a 1 L Erlenmeyer flask and dissolved with 240 ml of water; 360 mL of MeOH were added to this solution, obtaining a cloudy solution. This solution was placed in a high-pressure reactor with vigorous stirring, and 10 g of epoxide mixture (5) were added, the reactor was closed and heated at 95° C. for four hours, cooled to room temperature and carried out. CCD (Heptane-AcOEt (80:20) twice) developing with phosphomolybdic acid, then the reactor was placed in an ice bath for two hours and vacuum filtered. The solid obtained was dried in a vacuum oven at 45° C. for 4 hours and 4.83 g of sulfonate mixture (6) were obtained. The performance is reported in table 1.

### 3-alkoxy-2-hydroxypropane-1-sulfonate, Alternate Route (6B′)

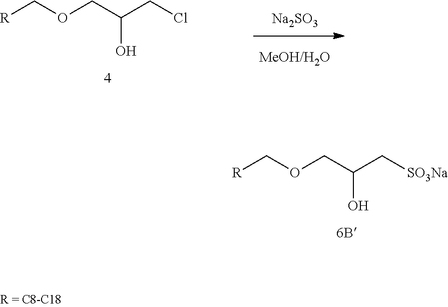

In a 1 L Erlenmeyer flask, 9.02 g (71.56 mmol) of Na2SO3 were placed and dissolved with 220 ml of water; 330 ml of MeOH were added, obtaining a cloudy solution. This solution was placed in a high-pressure reactor with vigorous stirring, and 10 g of chlorohydrin mixture (4) was added, the reactor was closed and heated at 95° C. for four hours. It was allowed to cool to room temperature and CCD was carried out (Heptane-AcOEt (80:20) twice) developing with phosphomolybdic acid, then the reactor was placed in an ice bath for two hours, vacuum filtered and washed with water. cold, the solid obtained was dried in a vacuum oven at 45° C. for 4 hours, obtaining 4.16 g of sulfonate mixture (6B′). The performance is reported in Table 1.

### General Scheme: Synthesis of Amphoteric Surfactants from Vegetable Oils

Vegetable oil (1) is used as starting material, where the chains R, R′, R″ can contain from 6 to 20 carbons with different degrees of unsaturation, some representative examples of vegetable oil include coconut, palm, palm kernel oil, jatropha curcas, castor, corn, canola, sunflower, soybean, birdseed, safflower, etc. Ammonia gas is generated and dissolved in methanol in a pressure reaction system to obtain the amide mixture (2A), then a reducing mixture of sodium borohydride with acetic acid is placed in refluxing tetrahydrofuran, thereby obtaining the mixture of amines (3A) that is treated with the Echweiler-Clarke reaction conditions to obtain the product 4A, subsequently, it is subjected to treatment with sodium chloroacetate in methanol to, finally,

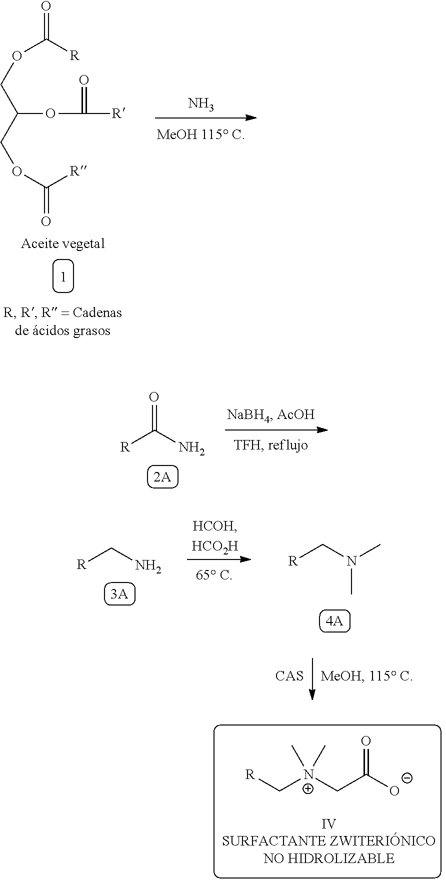

### Obtaining a Mixture of Amides (2A)

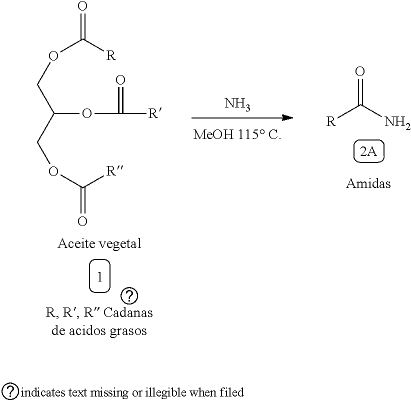

50 g of coconut oil, 400 mL of a 5.8M solution of dissolved dry ammonia were placed in a stainless-steel reaction vessel with a hermetic closure system equipped with an internal magnetic stirrer, manometer, and check valve with key for reagent supply. in methanol, 400 mg of NaCN were added as a catalyst, after the hermetic closure of the vessel, it was heated in an oil bath at 115° C.+/−1° C., for 10 hours. A representative aliquot is taken through the sampling valve, the progress of the reaction is verified by thin layer chromatography with a mobile phase Hexane: MTBE (92:8), it is revealed in an iodine chamber and bromocresol green, it is released the remaining gas to a recovery system, the reaction vessel is opened, vacuum filtered,

In this reaction, aminolysis of coconut oil is considered an affordable reaction in terms of atomic and environmental economics, therefore it can be carried out on an industrial scale, allowing controlled recovery of excess ammonia generated in situ and methanol.

### Obtaining Alkylamines by Reduction (3A)

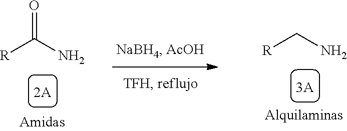

In a 1000 mL balloon flask, equipped with magnetic stirring and a moisture trap, 40 g of the 2A amide mixture were dissolved with 400 mL of anhydrous THF, 32 g (0.8 mol) of NaBH4 were added; The reaction mixture was stirred for 20 minutes at room temperature, then 48 g (0.8 mol) of AcOH were added, at the end of the time the system was cooled to 5° C., and 250 ml of a THF/AcOH (4:4) mixture were prepared. 1) and added to the reaction system through a stainless-steel cannula slowly at a rate of 1.25 mL per minute with temperature monitoring, the reaction is exothermic. At the end of the addition, it was kept in a refrigerator bath for 30 minutes and 20 minutes at room temperature. The mixture was refluxed for 4 h, the progress of the reaction was followed by thin layer chromatography with hexane mobile phase: ethyl acetate (1:1), run twice and using bromocresol green as developer. A solution of HCl (3%) was added to the reaction flask until a pH between 1 and 2 was obtained, to eliminate the excess of reducing agent, later 37.3 g of K2CO3 were added to release the amines until a pH between 8 and 9 was obtained, 400 ml of water were added and a liquid-liquid extraction was carried out with ethyl acetate (4×300 mL); the organic phase was dried over anhydrous sodium sulfate and concentrated to dryness, obtaining 37.9 g of the alkylamine mixture 3A. 3 g of K2CO3 to release the amines until a pH between 8 and 9 was obtained, 400 mL of water were added and a liquid-liquid extraction was carried out with ethyl acetate (4×300 mL); the organic phase was dried over anhydrous sodium sulfate and concentrated to dryness, obtaining 37.9 g of the alkylamine mixture 3A. 3 g of K2CO3 to release the amines until a pH between 8 and 9 was obtained, 400 mL of water were added and a liquid-liquid extraction was carried out with ethyl acetate (4×300 mL); the organic phase was dried over anhydrous sodium sulfate and concentrated to dryness, obtaining 37.9 g of the alkylamine mixture 3A.

In this reaction, a reducing agent with moderate chemical reactivity is used, with the purpose of generating a smooth, controlled and specific reaction, for this reason the formation of sodium acetoborohydride was used as a resource, because it allows the reaction to progress with a smaller amount. of reagent compared to the use of conventional sodium borohydride. In addition, acetoborohydride is a reagent that does not have a commercial presentation due to its stability and high speed of disproportionation-decomposition, which is why we generate it in situ, through the NaBH4-AcOH mixture, molar ratio (1:1), when treating of an exothermic reaction, care is taken that it does not reach temperatures higher than 20° C.

This reagent has a different chemical affinity compared to sodium triacetoxyborohydride, this modified species (sodium acetoborohydride) can reduce a wide variety of chemical species that are not normally associated or have low reactivity in borohydride chemistry. Without intending to limit themselves to any particular mechanism or theory, the inventors postulate that the foundation resides in the oxygen-boron bond, the oxygen of the acyloxy group having greater electronegativity compared to boron, tends to abstract electronic density from the medium, leaving more free to the hydride, converting it into a more reactive chemical species. FIG. 4 shows the structures of sodium borohydride (A), sodium acetoborohydride (B), sodium triacetoborohydride (C), the arrows indicate the direction of displacement of the electron density, the final part of the arrow that has a perpendicular cross line indicates the positive or deprotected region of the structure, as can be seen, structure A is totally symmetrical, structure C contains three acetoxy groups, therefore; theoretically it would have to be more reactive than structures A and B, however it presents less reactivity due to structural accommodation, the three acetoxy groups generate steric hindrance, which prevents a rapid reaction by the hydride but favors solubility in aprotic solvents however it is incompatible with methanol, moreover; compound B, having a single acetoxy group,

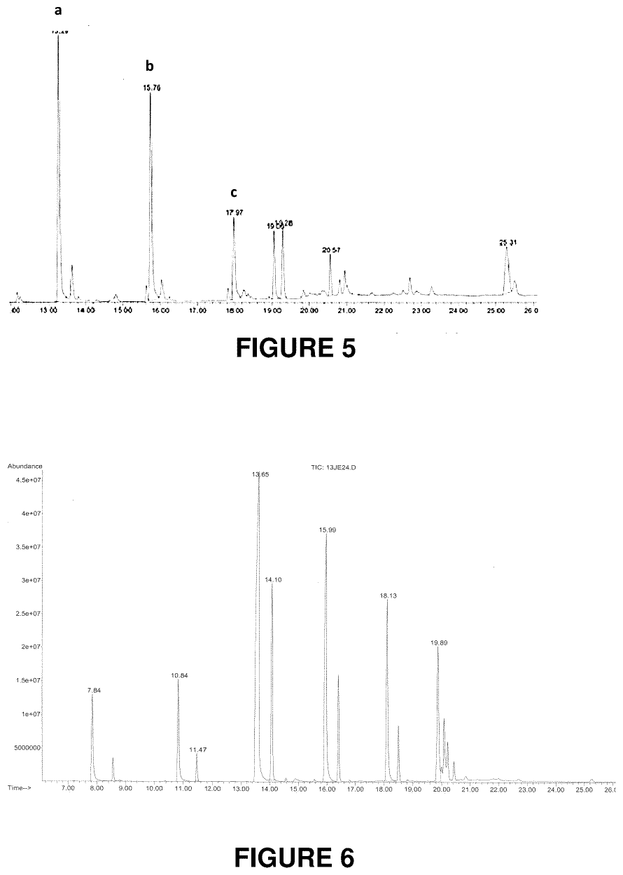

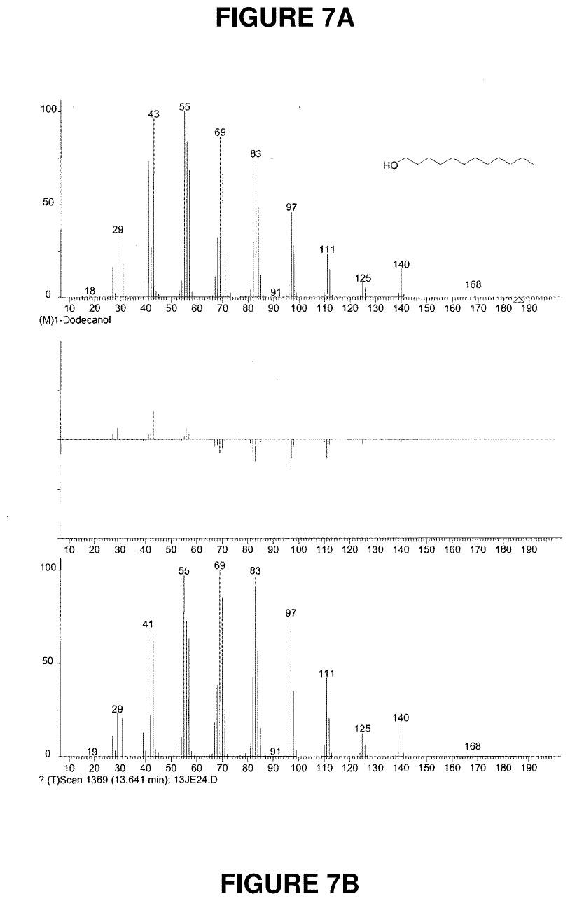

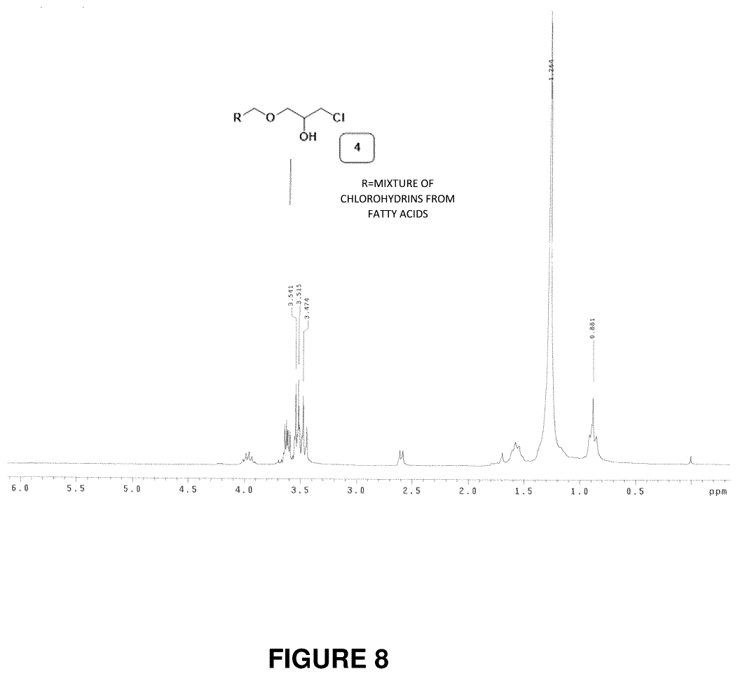

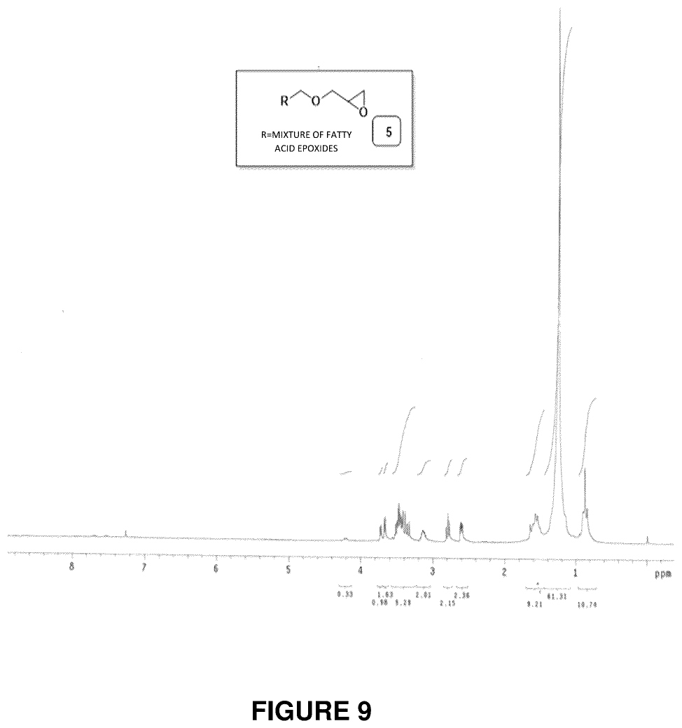

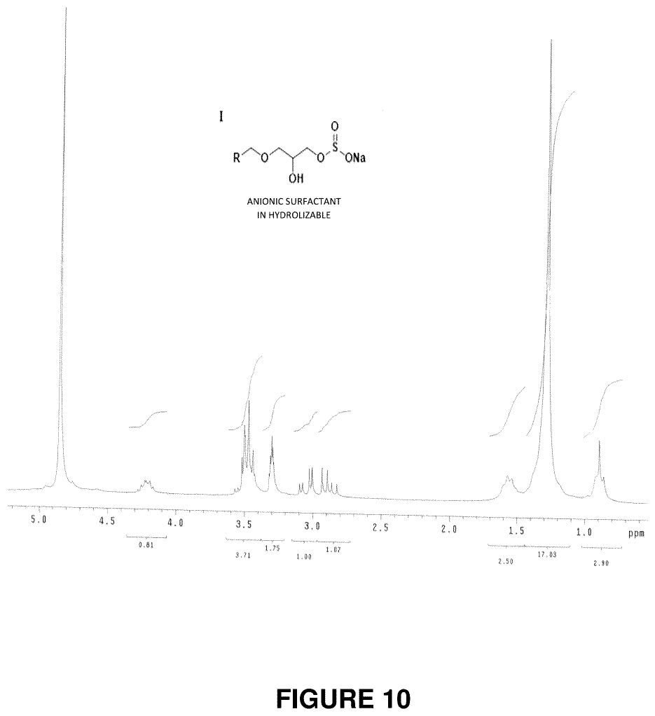

The mixture of alkylamines obtained (3A) was analyzed by gas chromatography (FIG. 5), the peak with a retention time of 13.22 minutes (a) corresponds to 12-carbon amines, the peak that appears at 15.70 minutes (b) corresponds to 14-carbon amines, while the 17.97 (c) peak corresponds to the 18 carbon amines, this includes the general mixture of alkylamines, the remaining peaks correspond to the different amines from 20 to 22 carbons, however they are found in a smaller proportion, all these peaks integrate and correspond to the mixture of C12-alkylamines. C18 as main components of raw material to obtain surfactants.

### Obtaining Alkyldimethylamines by Echweiler-Clarke Reaction (4A)

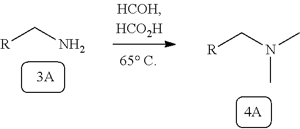

38 g of the product were placed in a 250 mL balloon flask. 3A, 44 mL (1 mol) of formic acid were slowly added, it was kept stirring for 10 minutes at room temperature to generate ammonium formates, then 44.1 mL of a formaldehyde solution (37%) were added and continued with stirring for 10 minutes, then the temperature was raised to 70° C. and allowed to react for 2 hours, at the end of the time the reaction was monitored through a thin layer chromatography plate with mobile phase DCM: AcOEt (90:10), when observing the total consumption of raw material and the presence of a slightly more polar product, a saturated NaHCO3 solution was added until a pH of 8 was obtained, and a liquid-liquid extraction was carried out with ethyl acetate in the form of a partition (3×30 mL), was separated and dried over anhydrous magnesium sulfate, The organic phase was concentrated in a rotary evaporator and 35.5 g of product were obtained. 4A.

### Obtaining Zwitterionic Surfactants (5a)

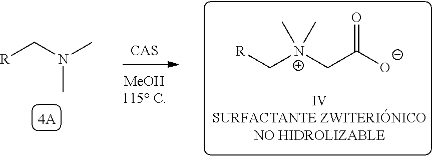

30 g of raw material were placed in a high-pressure reaction vessel equipped with a stirring system, sampling and injection valves, and a manometer. 4A, 300 mL of technical grade methanol and 16.40 g (0.14 mol) of sodium chloroacetate, was closed and hermetic was verified by pressurizing with 20 psi of nitrogen, the pressure was maintained for 30 minutes, it was released and heated to 110° C. for 6 hours, at the end of the established time, a representative sample was taken, a CCF plate was made in mobile phase DCM:MeOH:NH4OH (90:8:2), developed with iodine, vacuum filtered on a Solka-Floc bed, the liquid phase was concentrated until the first crystals were obtained, then the filter flask was taken to a MeOH: CO2 (S) refrigerator bath in static mode, the solid was filtered and 28.9 g of product were obtained. 5A.

Due to the structural nature of surfactants, it is conferred the property of being not hydrolyzable, this resides in the fact that structurally there is no functional group that presents chemical reactivity against this process, the hydrophilic segment is linked through a CN bond, added to this, the pair of nitrogen electrons is compromised with the carboxyl of the same site to give rise to the duality of formal charges (−, +) in the same segment, this nature adds electropositive character to nitrogen, therefore in electronegativity values on the Pauling scale the following values are given according to their radius atomic, Carbon: 2.5 Nitrogen 3.0, therefore it is a polar covalent bond, this difference of 0.5 electronegativity units gives it a hardness and to generate a CN bond excision, higher energy chemical conditions are needed.

In a preferential modality, the surfactants described in this specification can be included in compositions withone or more than one solvent and one or more than one additive for your application. Preferably in:

Anionic Surfactant
solvent system
solvent
cosolvent
paraffin and asphaltene inhibitor
surfactant of the invention

| | Amount | | |
|---|---|---|---|
| | (specific and | | Ingredient that can be |
| Ingredient | ranges) | Function | substituted |
| Solvent | Specific: 45% | Solubilize compounds of interest, be | Aroma 150 |
| | Range: 10-90% | the vehicle of compounds of interest | Xylene |
| | | in formulation | Toluene, |
| | | | Ethylbenzene |
| cosolvent | Specific: 25% | Create synergistic affect to solubilize | Butanol |
| (Butylcellosove) | Range: 10-40% | compound of interest with a smaller | Methylcellosolve |
| | | Volume | |
| Paraffin and | Specific: 5% | Avoid the formation of high molecular | Polyalkylsuccinimide |
| asphaltene | Range: 1-10% | weight hydrocarbon crystals | oxazolidinones |
| inhibitor | | | Oximes of aliphatic |
| | | | ketones. |
| surfactant | Specific: 25% | Lower the surface tension of the | Does not apply |
| | Range: 2-40% | aqueous phase to allow wettability of | |
| | | the rock | |

Cationic Surfactant or Mixture Cationic-
Zwitterionic Surfactant
Acid system (15%) delayed
water
iron sequestrant
dispersant
corrosion inhibitor
anti sludge
surfactant of the invention
formic acid
hydrochloric acid

| | Amount | | |
|---|---|---|---|
| | (specific and | | Ingredient that can |
| Ingredient | ranges) | Function | be substituted |
| Water | Specific: 44% | Solubilize compounds of interest, be | NA |
| | Range: 10-90% | the vehicle of compounds of interest | |
| | | in formulation | |
| iron sequester | Specific: 0.015% | remove dissolved iron | Polyphosphates |
| | Range: 0.01-2% | | EDTA |
| | | | Amino acids |
| dispersant | Specific: 2% | Avoid the formation of high | Long chain |
| | Range: 1-10% | molecular weight hydrocarbon | polyoxyethylenes |
| | | crystals | |
| corrosion | Specific: 2% | Reduce the speed of corrosion | Imidazolines |
| inhibitor | Range: 1-20% | caused by external agents | Amidoamides |
| anti slude | Specific: 2% | Avoid the formation of sludge in the | Sulfamic acid |
| | Range: 1-5% | stimulation process | |
| surfactant of the | Specific: 10% | Lower the surface tension of the | NA |
| invention | Range: 10-30% | aqueous phase to allow wettability | |
| | | of the rock | |
| Formic acid | Specific: 10% | Retard the reaction of hydrochloric | vinyl acid polymers |
| | Range: 5-20% | acid | |
| Hydrochloric | Specific: 29.95% | Rock material degrader. | NA |
| Acid 32% | Range: 20-40% | | |

Nonionic Surfactant
divergent system
water
potassium chloride
permeability improver
surfactant of the invention

| | Amount | | Ingredient |
|---|---|---|---|
| | (specific and | | that can be |
| Ingredient | ranges) | Function | substituted |
| Water | Specific: 91% | formulation vehicle | NA |
| | Range: 60-95% | | |
| Potassium | Specific: 2% | provide ions | Sodium chloride |
| chloride | Range: 1-10% | | |
| permeability | Specific: 2% | Allow diffusion in gaps | Bultilcellosolve |
| improver | Range: 1-10% | | methylcellosolve |
| surfactant | Specific: 5% | Lower the surface tension | NA |
| | Range: 2-10% | of the aqueous phase to | |
| | | allow wettability of | |
| | | the rock | |

### Examples

This example is illustrative and not limiting, since a person skilled in the art will understand that there are variants that fall within the scope of protection of the present invention.

TABLE 1
Yields of the synthesis of the various products obtained.

| | | | Melting | |
|---|---|---|---|---|
| Compound | Product | Appearance | point | Performance |
| 3A | fatty alcohol | solid white | 22° C. | NA (starting |
| | (dodecanol) | | | material) |
| 4A | chlorohydrin | Transparent | na | 93.50% |
| | | liquid | | |
| 5A | epoxy | Transparent | na | 83% |
| | | liquid | | |
| 6A | 3-dodecyloxy- | white flakes | 168° C. | 89% |
| | 2- | | | |
| | hydroxypropa | | | |
| | ne-1-sulfonate | | | |
| | compound | | | |
| 1 B | vegetable oil | solid white | 28° C. | NA (starting |
| | (coconut) | | | material) |
| 2B | mixture of | amber liquid | na | 93% |
| | methyl esters | | | |
| 3B | mixture of | amber liquid | na | 91% |
| | fatty alcohols | | | |
| 3 B′ | mixed fatty | amber liquid | na | 93% |
| | alcohols | | | |
| | (alternate | | | |
| | route b′) | | | |
| 4B | chlorohydrin | amber liquid | na | NA |
| | mixture | | | |
| 5B | apoxide mix | amber liquid | na | NA |
| 6B | anionic | pale flakes | na | 48% * |
| | surfactant | | | |
| 6 B′ | anionic | pale flakes | 171° | 42% * |
| | surfactant | | | |

* Calculated yieldfrom thecoconut oil.

### Efficiency Tests

The effectiveness of a surfactant is established based on various parameters related to its ability to solubilize hydrophobic compounds and decrease surface and/or interfacial tension. One of these parameters is the Critical Micellar Concentration or CMC, which is the minimum concentration of a surfactant agent capable of forming micelles. These micelles are produced when the hydrophobic part of the surfactant, being unable to form hydrogen bonds with water molecules, produces an increase in the free energy of the system. One way to alleviate this increase is to isolate the hydrophobic region by interacting with other surfaces, associating with other hydrophobic compounds or by forming vesicles (micelles) in which the lipophilic region is located in the center and the hydrophilic region on the outside.

The formation of mixed micelles between surfactants and other hydrophobic compounds such as hydrocarbons favor their dispersion in an aqueous medium, increasing bioavailability and consequently the possibility of degradation.

### Determination of Critical Micellar Concentration by UV-VIS Spectrometry

For the determination, a variant of the method described by Nasiru T. et al. was used, where the surfactant for which the critical micellar concentration (CMC) is to be determined is used, a dye solution 1-(2-pyridylazo)-2-naphthol (PAN) in pentanes and solutions of sodium chloride, nickel chloride, ferric chloride, among others. For this particular case, sodium lauryl sulfate was used as a reference, water as a blank, coconut surfactants, lauric alcohol, and bromophenol blue as a dye.

The changes in the CMC refer to the measurement of the absorbance of the dye-surfactant mixture and is given as a function of the surfactant concentration, which makes it possible to quantitatively determine the CMC of each sample to be determined. Specifically, at concentrations below the CMC, absorption is extremely low. However, when the CMC is ideal, there is a sudden increase in absorbance.

Above the CMC, the absorbance of the solution increases linearly with increasing concentration. The CMC occurs at the concentration where the two lines intersect, or when the flat absorption line begins to increase.

100 ml of a 1.6×10-3 M bromophenol blue solution in 96° ethanol were prepared, solutions of 100, 200, 300, 400, 500, 600, 700, 800, 900 and 1000 ppm of sodium lauryl sulfate (reference) and another ten at the same concentrations, but with a mixture of anionic, cationic, non-ionic and zwitterionic surfactants, all of them in water.

A study with different “scanning” wavelengths was carried out to find the maximum absorption wavelength for bromophenol blue with a concentration of 1.6×10-3 M, the results are shown in the following table:

TABLE 2
Data for bromophenol blue wavelength scan and graph.

| absorbance | Wavelength □ |
|---|---|
| 0.01 | 200 |
| 0.0587 | 221 |
| 0.1169 | 301 |
| 0.1786 | 395 |
| 0.3122 | 402 |
| 0.3245 | 428 |
| 0.3469 | 512 |
| 0.9026 | 562 |
| 1.1237 | 592 |
| 0.3750 | 664 |
| 0.2290 | 729 |
| 0.1087 | 803 |
| 0.0821 | 821 |

From the results shown in the previous table it is clear that the maximum absorption value for bromophenol, with a concentration of 1.6×10-3 M, is 592 nm. For this reason, in the studies to determine the critical micellar concentration, the wavelength of 592 nm was used; for which the corresponding mixtures were prepared by adding 1 mL of each surfactant solution plus 1 mL of bromophenol blue solution (1.6×10-3M). The results obtained are shown in the following table:

TABLE 3
Absorbance of the non-hydrolyzable surfactant solutions of the present invention.

| LSS | ABS | ABS | CATIONIC | ABS | ZWITTERIONIC |
|---|---|---|---|---|---|
| (PPM) | REFERENCE | ANIONIC | ABS | NON IONIC | ABS |
| 0 | 0 | 0 | 0 | 0 | 0 |
| 100 | 0.1121 | 0.1091 | 0.8079 | 0.097 | 0.2871 |
| 200 | 0.1226 | 0.1194 | 0.9451 | 0.101 | 0.3055 |
| 300 | 0.2098 | 0.2063 | 0.9762 | 0.1526 | 1.6801 |
| 400 | 1982 | 0.7925 | 1.9989 | 0.1834 | 1.7237 |
| 500 | 2.0187 | 0.9802 | 2.1127 | 0.2742 | 1.8899 |
| 600 | 2.3647 | 2.0351 | 2.2682 | 0.3245 | 2.109 |
| 700 | 2.4687 | 2.2284 | 2.3498 | 0.4266 | 2.2958 |
| 800 | 2.4799 | 2.258 | 2.402 | 1.6888 | 2.4087 |
| 900 | 2.5588 | 2.3566 | 2.6345 | 1.7247 | 2.6245 |
| 1000 | 2.6001 | 2.1087 | 2.721 | 1.909 | 2.9483 |

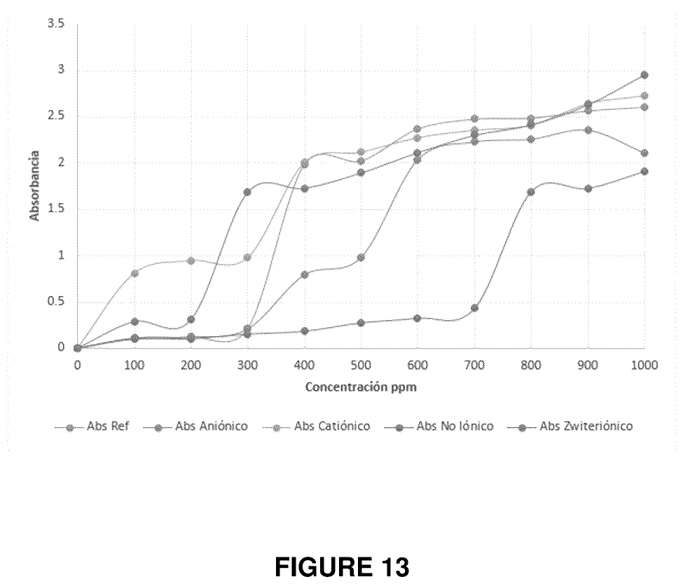

As can be seen in the graph of FIG. 13, and its corresponding table 4, the critical micelle concentration refers to the minimum effective concentration to form micelles. The appropriate amounts for each surfactant are shown below.

TABLE 4
Critical micellar concentration. of the
surfactants of the present invention.

| | Critical micellar concentration | |
|---|---|---|
| surfactant | (ppm) | absorbance |
| Reference | 400 | 1982 |
| anionic | 600 | 2.0351 |
| cationic | 400 | 1.9989 |
| non ionic | 800 | 1.6888 |
| zwitterionic | 300 | 1.6801 |

### Determination of the Stability to Hydrolysis of Surfactants.

Surfactants were designed with the main characteristic of maintaining their chemical structure during and after being subjected to high temperatures, specifically at the working temperature that occurs in an oil well. For which a work simulation was implemented as presented in an oil well, temperature of 180° C. with a pressure of 4000 psi in a nitrogen atmosphere in an acidified medium with 1M HCl.

A 10% solution in distilled water was prepared, 100 ml of this solution plus 25 mL of hexane was placed in a stainless steel beaker, purged with nitrogen 5 times, hermetically sealed, heated to 180° C. and the pressure was adjusted to 4000 psi with controlled addition of nitrogen, it was kept stirring for 12 hours, at the end of the time, the aqueous phase was extracted, traces of organic solvent were eliminated by heating under reduced pressure, 1 mL of and HPLC high performance liquid chromatography analysis was performed on a reverse phase column.

The chromatography study was carried out using an AGILENT 1200 chromatograph, C18 reverse phase column, model XChroma HPLC C18-aqueous, 5 μm×4×150 mm, mobile phase MeOH-MeCN—H2O ratio 4:4:2, wavelength reading distance 254 nm, working temperature 32° C., flow rate 0.9 mL/min and injected quantity of 5 μL.

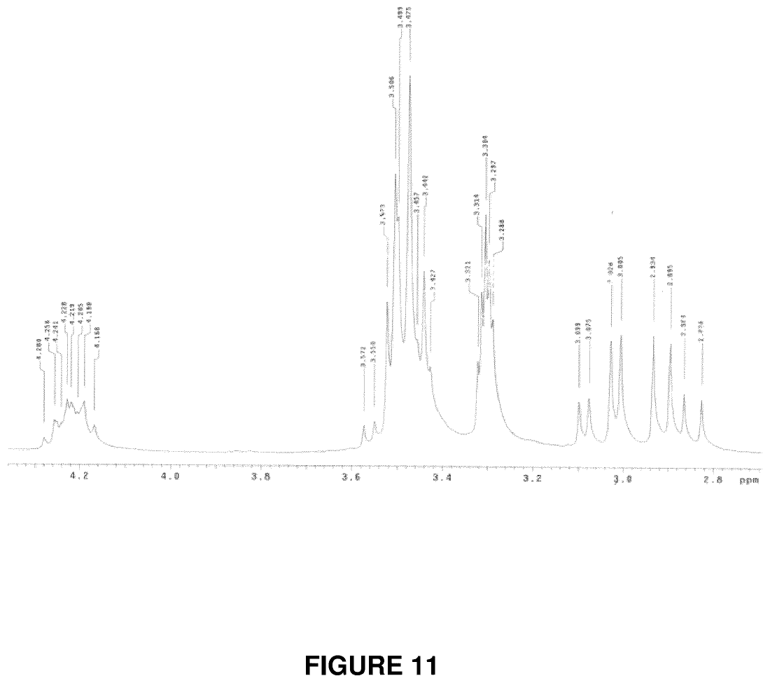

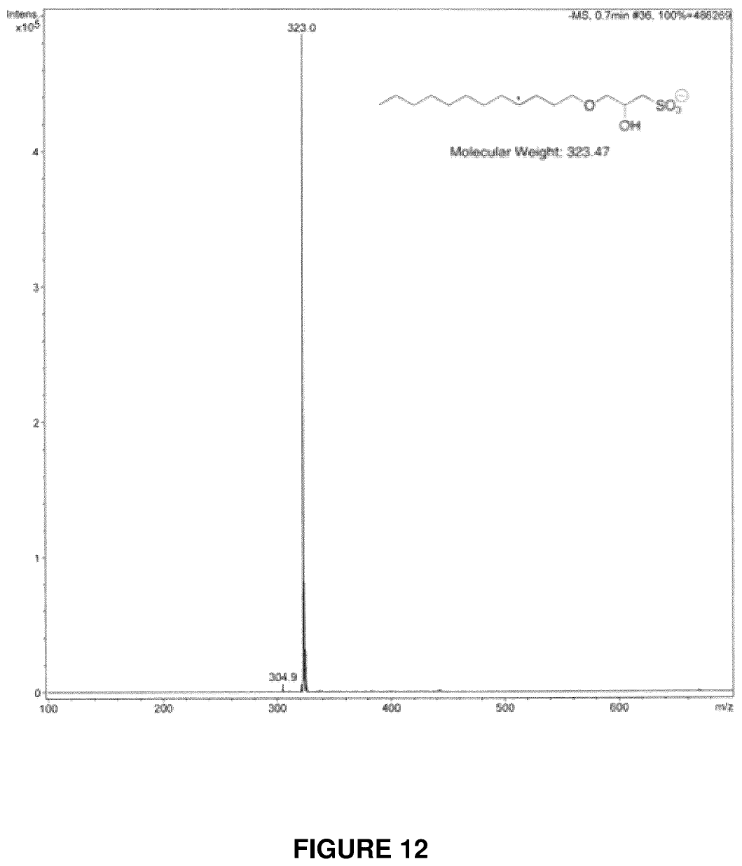

anionic surfactant: Two injections were made for each surfactant, the first as a reference and the second as experimental. The values obtained from the chromatography study are shown in Table 5 and 6, especially peaks 11 and 12 of each test. On the other hand, the chromatograms, before and after the stability experiment, are shown in FIG. 11.

TABLE 5
reference test

| Peak | RetTime | | Width | Area | Height | Area |
|---|---|---|---|---|---|---|
| # | [min] | Type | [min] | [mAU*s] | [mAU] | % |
| 1 | 3.331 | BV | 0.0324 | 12.76394 | 5.75785 | 0.1374 |
| 2 | 3.404 | VV | 0.0421 | 7.51850 | 2.57063 | 0.0809 |
| 3 | 3.454 | VB | 0.0327 | 7.04310 | 3.19548 | 0.0758 |
| 4 | 3.593 | BB | 0.0557 | 8.10312 | 2.09241 | 0.0872 |
| 5 | 4.004 | BB | 0.3259 | 47.98637 | 1.72838 | 0.5166 |
| 6 | 4.528 | BB | 0.0931 | 22.08399 | 2.82885 | 0.2377 |
| 7 | 5.237 | BV E | 0.1234 | 16.38464 | 1.57401 | 0.1764 |
| 8 | 5.459 | VV R | 0.1339 | 100.76517 | 10.18454 | 1.0848 |
| 9 | 5.698 | VB | 0.1660 | 136.04500 | 11.54663 | 1.4646 |
| 10 | 6.240 | BB | 0.1202 | 38.23884 | 3.83815 | 0.4117 |
| 11 | 8.320 | BB | 0.4041 | 4406.66846 | 157.88963 | 47.4404 |
| 12 | 10.157 | BB | 0.5450 | 4485.25488 | 98.17143 | 48.2864 |

| | | |
|---|---|---|
| Totals: | 9288.85601 | 301.37801 |

TABLE 6
Experimental test, extreme conditions of temperature and pressure.

| Peak | RetTime | | Width | Area | Height | Area |
|---|---|---|---|---|---|---|
| # | [min] | Type | [min] | [mAU*s] | [mAU] | % |
| 1 | 3.333 | BV | 0.0311 | 12.85824 | 6.09056 | 0.3310 |
| 2 | 3.406 | VV | 0.0413 | 7.50910 | 2.58934 | 0.1933 |
| 3 | 3.456 | VB | 0.0327 | 7.36634 | 3.35025 | 0.1896 |
| 4 | 3.593 | BB | 0.0594 | 9.12915 | 2.22273 | 0.2350 |
| 5 | 4.014 | BB | 0.1217 | 18.02070 | 1.74872 | 0.4639 |
| 6 | 4.528 | BB | 0.1538 | 14.08968 | 1.08264 | 0.3627 |
| 7 | 5.460 | VV | 0.1107 | 34.41470 | 3.91021 | 0.8859 |
| 8 | 5.697 | VB | 0.1644 | 142.26663 | 10.51218 | 3.6623 |
| 9 | 6.254 | BB | 0.0963 | 8.24784 | 1.02061 | 0.2123 |
| 10 | 6.517 | BB | 0.2021 | 40.86639 | 2.38215 | 1.0520 |
| 11 | 8.322 | BB | 0.3685 | 1803.33191 | 64.23315 | 46.4223 |
| 12 | 10.158 | BB | 0.5363 | 1786.52625 | 39.01283 | 45.9896 |

| | | |
|---|---|---|
| Totals: | 3884.62693 | 138.15530 |

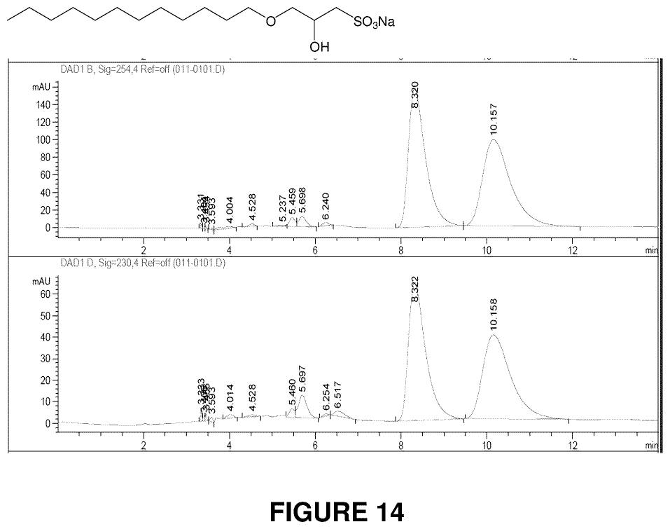

In FIG. 14, both chromatograms are shown, the upper section 1 (table 5) corresponds to the reference test, in which two characteristic peaks are observed with a retention time of 8,320 min and 10,157 min, this is due to the composition of surfactant formulation. While test 2 (table 6) corresponds to the analysis of the anionic surfactant after being subjected to extreme conditions of pressure and temperature (4000 psi and 180° C.), the chromatogram shows the same retention peaks at 8,320 min and 10,157 min, the appearance of signals at 6,517 min and disappearance of the signal at 5,237 min is also observed, however, the majority peaks remain, indicating that surfactant is potentially steady against extreme working conditions.

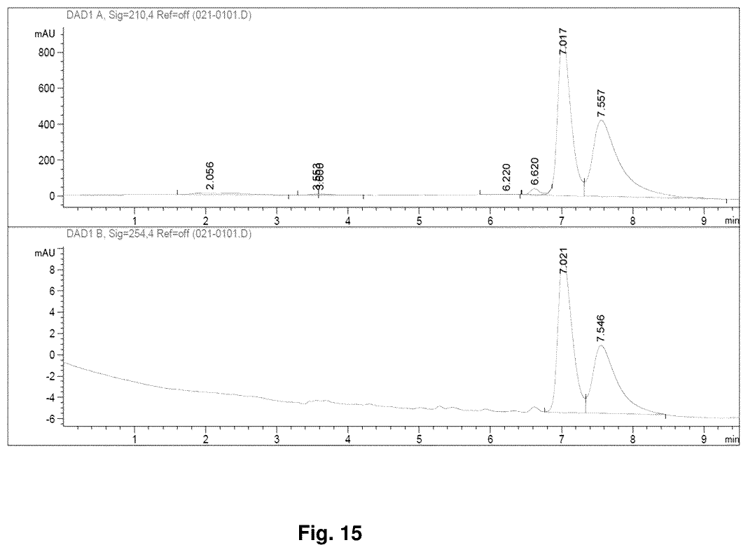

Cationic surfactant: Two injections were made for each surfactant, the first as a reference and the second as experimental, the values obtained from the chromatography study are shown in table 7 (peaks 6 and 7) and 8 (peaks 1 and 2), the chromatograms, before and after the stability experiment are shown in FIG. 15.

TABLE 7
reference test

| Peak | RetTime | | Width | Area | Height | Area |
|---|---|---|---|---|---|---|
| # | [min] | Type | [min] | [mAU*s] | [mAU] | % |
| 1 | 2.056 | BB | 0.6185 | 539.68134 | 10.24582 | 2.2605 |
| 2 | 3.553 | BV | 0.0908 | 55.10683 | 7.27774 | 0.2308 |
| 3 | 3.600 | VB | 0.1460 | 88.65709 | 7.23263 | 0.3713 |
| 4 | 6.220 | BB | 0.1850 | 19.85797 | 1.26598 | 0.0832 |
| 5 | 6.620 | BV E | 0.1381 | 324.98303 | 34.44081 | 1.3612 |
| 6 | 7.017 | VV R | 0.1932 | 1.13752e4 | 889.48328 | 47.6454 |
| 7 | 7.557 | VBA | 0.3779 | 1.14712e4 | 425.00143 | 48.0476 |

| | | |
|---|---|---|
| Totals: | 2.38747e4 | 1374.94769 |

TABLE 8
Experimental test, extreme conditions of temperature and pressure.

| Peak | RetTime | | Width | Area | Height | Area |
|---|---|---|---|---|---|---|
| # | [min] | Type | [min] | [mAU*s] | [mAU] | % |
| 1 | 7.021 | BV | 0.1790 | 202.73399 | 14.83161 | 56.9873 |
| 2 | 7.546 | VB | 0.2811 | 153.01872 | 6.38393 | 43.0127 |

| | | |
|---|---|---|
| Totals: | 355.75272 | 21.21554 |

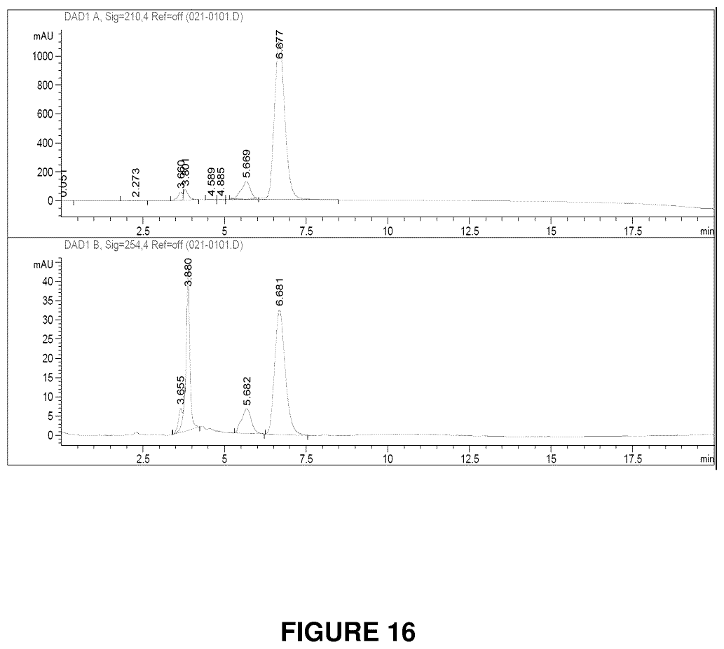

Nonionic Surfactant: Two injections were made for each surfactant, the first as a reference and the second as experimental, the values obtained from the chromatography study are shown in table 9 (peak 8) and 10 (peaks 2 and 4), the chromatograms, before and after the stability experiment are shown in FIG. 16.

TABLE 9
reference test

| Peak | RetTime | | Width | Area | Height | Area |
|---|---|---|---|---|---|---|
| # | [min] | Type | [min] | [mAU*s] | [mAU] | % |
| 1 | 0.051 | BB | 0.1261 | 12.49840 | 1.20454 | 0.0431 |
| 2 | 2.273 | BB | 0.1935 | 47.57958 | 2.89138 | 0.1639 |
| 3 | 3.660 | BV | 0.1420 | 555.57910 | 58.64502 | 1.9144 |
| 4 | 3.801 | VB | 0.1319 | 718.83081 | 74.91354 | 2.4769 |
| 5 | 4.589 | BV | 0.1681 | 30.33521 | 2.15008 | 0.1045 |
| 6 | 4.885 | VB | 0.1153 | 21.46611 | 2.30087 | 0.0740 |
| 7 | 5.669 | BV E | 0.2799 | 2358.36548 | 119.67072 | 8.1265 |
| 8 | 6.677 | VB R | 0.3348 | 2.52762e4 | 1115.66016 | 87.0967 |

TABLE 10
Experimental test, extreme conditions of temperature and pressure.

| Peak | RetTime | | Width | Area | Height | Area |
|---|---|---|---|---|---|---|
| # | [min] | Type | [min] | [mAU*s] | [mAU] | % |
| 1 | 3.655 | BV E | 0.1135 | 38.58822 | 4.73843 | 3.0876 |
| 2 | 3.880 | VB R | 0.1178 | 351.17096 | 42.54030 | 28.0983 |
| 3 | 5.682 | BB | 0.2464 | 133.35149 | 6.39196 | 10.6699 |
| 4 | 6.681 | BB | 0.2740 | 726.68530 | 32.35804 | 58.1443 |

| | | |
|---|---|---|
| Totals: | 1249.79597 | 86.02874 |

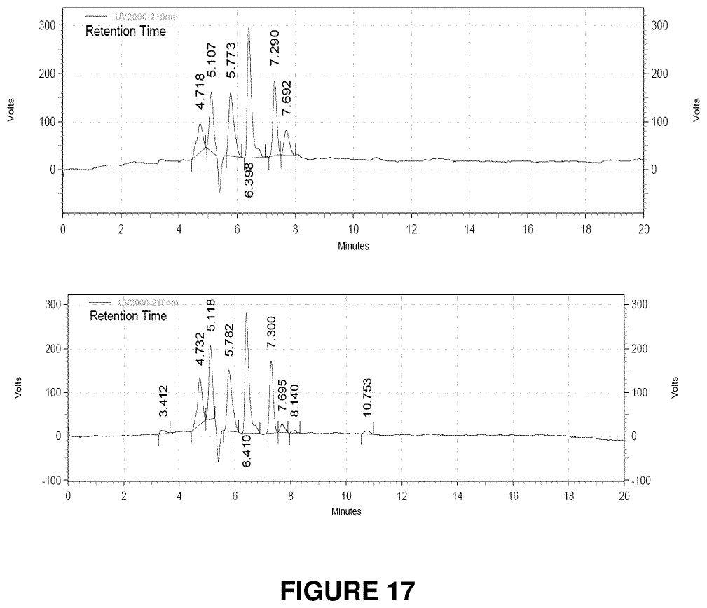

Zwitterionic surfactant: Two injections were made for each surfactant, the first as a reference and the second as experimental. The values obtained from the chromatography study, before and after the stability experiment, are shown in FIG. 17.

The study was carried out with the WATERS 1525 chromatograph, with the same experimental conditions and the same column previously indicated. The chromatograms are shown in FIG. 17. The first analysis, upper chromatogram, shows a major peak with a retention time of 6,398 minutes, the other signals correspond to components of the formulation. The lower chromatogram corresponds to the experimental test, under critical conditions of pressure and temperature, in both chromatograms the same signals are shared, which shows that there was no decomposition of the surfactants analyzed and/or alteration in the tested formulation vs. the reference one.

Notwithstanding the fact that the above description was made taking into account the preferred embodiments of the invention, those skilled in the art should take into account that any modification in shape and detail will be within the spirit and scope of the present invention. The terms in which this report has been drafted should always be taken in a broad and non-limiting sense. The materials, shape and description of the elements will be subject to variation if this does not imply an alteration of the essential characteristic of the invention.

1. A chemical synthesis process to obtain non-hydrolyzable surfactants comprising the following steps:
a) treating vegetable oils with potassium hydroxide and methanol, for the formation of the mixtures of methyl esters of the corresponding fatty acids;
b) the methyl esters, from the previous stage, are treated with sodium borohydride (NaBH4), tetrahydrofuran (TFH) in methanol to generate the corresponding fatty alcohol mixture;
c) the fatty alcohols, from the previous stage, are treated with zinc chloride and epichlorohydrin to generate the corresponding fatty acid chlorohydrin mixture; at the same time
d) the fatty acid chlorohydrin mixture is treated with potassium hydroxide in methanol to generate the fatty acid epoxide mixture, and
e) Chemical synthesis is directed from the epoxide to obtain anionic, cationic or non-ionic non-hydrolyzable surfactants.

2. The chemical synthesis process for obtaining non-hydrolyzable surfactants in accordance with claim 1, wherein the fatty alcohols, from the stage of subparagraph c), are obtained directly from vegetable oil, in the presence of tetrahydrofuran (TFH) and sodium borohydride. sodium (NaBH4) at 50° C. for 10 min, slowly add methanol and reflux for two hours to obtain the mixture of fatty alcohols.

3. The chemical synthesis process for obtaining non-hydrolyzable surfactants in accordance with claim 1, wherein the anionic non-hydrolyzable surfactants are obtained by reacting the fatty acid epoxide, step e) with Na2SO3 and methanol in water.

4. The chemical synthesis process for obtaining non-hydrolyzable surfactants in accordance with claim 1, wherein the cationic non-hydrolyzable surfactants are obtained by reacting the fatty acid epoxide, step e) with diethylenetriamine, is leave stirring for 1 hour. Next, anhydrous ethyl ether is added, HCL gas is bubbled in until white precipitates are obtained to obtain the cationic surfactant.

5. The chemical synthesis process for obtaining non-hydrolyzable surfactants in accordance with claim 1, wherein the non-ionic non-hydrolyzable surfactants are obtained by reacting the fatty acid epoxide, step e) with diethanolamine, is heated to 50° C. During 4 hours; and purified by a percolated column of silica gel in phase with dichloromethane to obtain the nonionic surfactant.

6. The chemical synthesis process for obtaining non-hydrolyzable surfactants in accordance with claim 1, wherein the non-hydrolyzable surfactants are obtained directly from the mixture of fatty acid chlorohydrins, step of item d). Na2SO3 is placed and dissolved in water; MeOH was added and added to the chlorohydrin mixture, heated at 95° C. for 4 hours, cooled for 2 hours on ice and non-hydrolyzable surfactants were obtained.

7. A chemical synthesis process to obtain non-hydrolyzable surfactants comprising the following steps:
a) vegetable oils are treated with dry ammonia dissolved in methanol and NaCN as a catalyst for the formation of the amide mixtures of the corresponding fatty acids;
b) the amides, from the previous stage, are treated with anhydrous tetrahydrofuran (TFH), sodium borohydride (NaBH4) and acetic acid, kept stirring for 20 min at room temperature; a THF/AcOH (4:1) mixture is added. The mixture is brought to reflux, the pH is adjusted between 1 and 2 and K2CO3 is added to obtain the alkylamine mixture;
c) the alkylamines, from the previous stage, are treated with formic acid to generate ammonium formates, formaldehyde is added, it is allowed to react at 70° C. until the reaction is complete, a saturated NaHCO3 solution is added until a pH of 8 is obtained, and
d) The product from the previous stage is treated with sodium chloroacetate and methanol at a pressure of 20 psi for 30 min, the pressure is released and it is heated at 110° C. for 6 hrs. to generate amphoteric non-hydrolyzable surfactants.

8. The non-hydrolyzable surfactant obtained in accordance with the synthesis processes according to claim 1, which is selected from the following group: 2,2′-((3-dodecyloxy)-2-hydroxypropyl) azadienyl)bis(ethan-1-ol); N,N-bis(2-aminoethyl)-3-(dodecyloxy)-2-hydroxypropan-1-ammonium; Sodium 3-dodecyloxy-2-hydroxypropan-1-sulfonate, 2-dodecyldimethylammonium acetate, and any combination of thereof.

9. A drilling fluid composition comprising one or more of the surfactants of claim 8, wherein one or more is a solvent and one or more is an additive.

10. The drilling fluid composition according to claim 9, wherein the one or more of the surfactants is in an amount ranging from 1 to 95% by weight.

11. The drilling fluid composition according to claim 8, wherein the additive is selected from a group consisting of a solvent, a cosolvent, a paraffin and asphaltene inhibitor, an iron sequestering agent, a dispersant, a corrosion inhibitor, an acid, a salt, an anti-sludge, and a permeability improver.
